(19) United States
(12) Patent Application Publication
(10) Pub. No.: US 2011/0137147 A1
(43) Pub. Date: Jun. 9, 2011

(54) MINIMUM TIME FEEDBACK CONTROL OF EFFICACY AND SAFETY OF THERMAL THERAPIES

(75) Inventors: Mikhail Skliar, Salt Lake City, UT (US); Robert Roemer, Summit Park, UT (US); Dhiraj Arora, Niskayuna, NY (US)

(73) Assignee: UNIVERSITY OF UTAH RESEARCH FOUNDATION, Salt Lake City, UT (US)

(21) Appl. No.: 12/089,597

(22) PCT Filed: Oct. 10, 2006
(86) PCT No.: PCT/US06/39505

§ 371 Date: Oct. 7, 2008

Related U.S. Application Data
(60) Provisional application No. 60/726,673, filed on Oct. 14, 2005.

(51) Int. Cl. A61B 5/055; A61B 18/00

(52) U.S. Cl. 600/411; 607/96

(57) ABSTRACT

A thermal treatment control system including an imaging device for specifying the geometry and/or location of the treatment target, a thermal energy element for applying a thermal treatment for the heating or cooling of a target tissue for therapeutic purposes, a thermal energy detecting element for detecting a measured tissue response to the thermal treatment and a feedback controller for a real-time modification of the intensity and spatial distribution of the thermal dose in order to achieve therapeutic efficacy over a minimum or reduced treatment time while satisfying treatment constraints imposed to limit damage to normal tissues.

### PRIORITY CLAIM

This application claims the benefit under 35 U.S.C. §119(e) of U.S. Provisional Patent Application Ser. No. 60/726,673, filed Oct. 14, 2005, for “MINIMUM TIME FEEDBACK CONTROL OF EFFICACY AND SAFETY OF THERMAL THERAPIES,” the entire contents of which are hereby incorporated herein by this reference.

### STATEMENT REGARDING FEDERALLY SPONSORED RESEARCH OR DEVELOPMENT

This invention was made with government support under grant #NCI-R01-CA33922 awarded by the National Institutes of Health, and under grant #CTS 0117300 awarded by the National Science Foundation. The Government has certain rights to this invention.

### TECHNICAL FIELD

The present invention relates generally to medicine and the thermal treatment of tumor tissues. More particularly, the invention relates to methods and apparatus of a treatment control system for invasive and noninvasive heating or cooling of target tissues for therapeutic purposes using ultrasound (US), radiofrequency (RF), microwave or other means of heating wherein the treatment control system is capable of real-time modification of the intensity and spatial distribution of tissue heating or cooling in order to maximize treatment efficacy and limit damage to normal tissues in reduced or minimum treatment time.

### BACKGROUND

Thermal therapies that utilize cooling or heating with different modalities such as ultrasound, radiofrequency or microwave may be used for treatment of different conditions, including various cancer types and in various anatomical sites such as brain, prostate and breast. Moreover, thermal therapies may be used to ablate tumors and coagulate target tissues without the use of invasive surgeries and procedures. For example, focused ultrasound therapies to noninvasively ablate spatially distributed targets inside patients are well known in the art and have been demonstrated in several studies, e.g., Poissonnier et al. 2003, Uchida et al. 2002, Wu et al. 2001 and Hynynen et al. 1991, which are incorporated herein by reference. Thermal therapies may also be used as an adjuvant modality in conjunction with radiation and chemotherapy as known by those of skill in the art, e.g., Bornstein et al. 1993 and Guthkelch et al. 1991, incorporated herein by reference.

From the clinical perspective, the goal of thermal therapies is to achieve a desired thermal dose distribution in a target tissue without threatening the safety of critical healthy tissues. A thermal dose quantifies the relationship between treatment efficacy and the target temperature as a function of time, where treatment efficacy depends on the cumulative effects of heating or cooling of the target tissue over the treatment time. The lack of adequate control of thermal therapies results in long treatment times, incomplete treatment of large targets, and unintended normal tissue damage, thus impedes a broader penetration of thermal therapies into clinical practice.

Accordingly, it would be advantageous to have an automatic treatment control system that automatically delivers in minimum or reduced treatment time the desired thermal dose to the target without damaging normal tissues, in the presence of treatment disturbances such as changing blood flow and thermal energy absorption or dissipation.

### DISCLOSURE OF INVENTION

One embodiment of a control system that automatically delivers a physician-prescribed thermal dose in minimum time without violating the imposed normal tissue constraints was described by those of skill in the art as discussed in Arora et al. 2005a, Arora et al. 2005b, Arora et al. 2005c, Arora et al. 2004, Arora et al. 2003, Palussiere et al. 2003, Arora et al. 2002 and Arora et al. 2006, all of which are incorporated herein by reference. Compared to the traditional approach, a real-time automatic feedback treatment control system would offer a number of advantages, including but not limited to: (1) robustness with respect to patient-to-patient variability and various treatment disturbances, such as changes in temperature-dependent ultrasound absorption and tissue perfusion; (2) normal tissue safety; (3) direct control of the thermal dose; and (4) reduced treatment time. As such, it would be desirable to provide an automatic thermal treatment control system that delivers a desired thermal dose distribution to the target tissue in a minimum time without causing healthy tissue damage and minimize patient pain and discomfort.

The present invention provides an automatic thermal control system characterized by a means to translate clinical efficacy and safety goals of thermal therapies into achievable automatic control objectives. The system includes an imaging device for specifying the geometry and/or location of the treatment target, a thermal energy element for applying a thermal treatment wherein the thermal treatment comprises heating or cooling of a target tissue for therapeutic purposes, a thermal energy detecting element for detecting a measured tissue response to the thermal treatment, and a feedback controller for a real-time modification of the intensity and spatial distribution of the applied thermal treatment to either (a) deliver the desired thermal dose in a minimum or reduced treatment time, or (b) to maintain the temperature of the target tissue as close to the desired value as possible, and (c) achieving efficacy goals (a) or (b) without violating safety constraints explicitly imposed in the selected normal tissue locations.

The thermal treatment control system may further comprise data processing means that collects signals or data output from the imaging device, the thermal energy element, the thermal energy detecting element, and the feedback controller, and thus coordinates the actions of these elements of the thermal treatment control system.

The feedback controller may include a predictive thermal model. The predictive model may include a transducer model, which predicts what will be the energy distribution of a transducer for given values of inputs that can be manipulated. The energy is often given in terms of a specific absorption rate (SAR). For example, such a transducer model can predict the SAR in a patient for a given electrical input to a transducer.

In one embodiment, the invention may allow one to use appropriate techniques to specify: (a) the prescribed target treatment efficacy in terms of a thermal dose, a temperature change, a temperature distribution or other treatment characterization which are delivered to the target to achieve therapeutic outcome; (b) treatment constraints that ensure safety of the tissues outside the specified treatment target, which may be in the form of temperature, temperature change, thermal dose and/or limits on the thermal response of the normal tissues in specified locations; and (c) hardware constraints of a given thermal treatment equipment, which may include limits on maximum applicable power or location of an applicator. The geometry and/or location of the treatment target may be specified by an appropriate technique, e.g., by an imaging device.

In another embodiment, the invention includes invasive or noninvasive means of heating or cooling of target tissue for therapeutic purposes. Examples of noninvasive power delivery include ultrasound, RF or microwave heating using a single or multiple transducers, or transducer arrays. Examples of invasive means of heating include an interstitial RF needle and/or optical laser applicators. Time-dependent power delivery or removal may be characterized by a spatial distribution and intensity, which may be manipulated during the treatment. In a particular embodiment, the noninvasive means of heating is a focused ultrasound heating system that is compatible with magnetic resonance (MR).

Another embodiment of the invention discloses means to measure tissue response to the delivered or removed energy. Such measurements may be invasive and require introduction of probes, or noninvasive such as magnetic resonance temperature measurements.

In a particular embodiment, the invention includes a MR thermometry feedback. Noninvasive thermal images obtained by MR may be used during a pre-treatment heating sessions to characterize spatial distributions of applied power, effective blood perfusion, and thermal response of the tissues to thermal excitation. Noninvasive MR thermal images may also be used for online feedback control of target thermal dose.

In a further embodiment, the invention includes a treatment control system capable of automatically modifying the intensity and spatial distribution of tissue beating or cooling for the purpose of thermal treatment without violating normal tissue and other safety constraints as disclosed. The measured tissue response to the treatment may be used as the feedback of the control system in order to modify the treatment evolution in real time to achieve efficacy and safety objectives while minimizing the treatment time. In a particular embodiment, the temperature control system comprises a thermal dose controller, which may be a nonlinear thermal dose controller. The thermal dose controller calculates the dose deficit as the difference between a desired dose and the already delivered thermal dose which is estimated based on the temperature measurements, and uses the thermal dose deficit as a feedback in the dose controller to continuously generate a reference temperature trajectory for a secondary temperature controller. The secondary temperature controller may be a linear, constrained, model predictive controller, which uses temperature measurements as a feedback and finds a heating power that minimizes the difference between the reference temperature and the temperature achievable without violating normal tissue safety constraints. An appropriate heating power found by the secondary temperature controller is applied to the target location of a subject. The combination of the main thermal dose controller and the secondary constrained temperature controller is such that the overall treatment control system provides direct, time-optimal feedback control of a thermal dose, thus a desired thermal dose is delivered to a target while limiting the peak temperature in a operator-selected normal tissue locations below a specified value, which is low enough to prevent normal tissue damage.

The invention may be applicable in the cases of: (a) a single stationary transducer, such as a stationary focused US transducer or an interstitial RF needle; (b) a single transducer, which may be scanned or repositioned by mechanical or other means; (c) multiple stationary transducers, such as a stationary phased array of individual controlled ultrasound transducers, or multiple RF needles; (d) multiple transducers that can be scanned or repositioned by mechanical or other means and (e) any combination of the above. The treatment control system can also account for and control an active cooling of the normal tissues, provided by surface (e.g., skin) or interstitial cooling

A further embodiment of the invention discloses a model-based operation of the control system.

A still further embodiment of the invention discloses an adaptive control of the treatment, including dynamic re-identification of the treatment and transducer models.

A yet still further embodiment of the invention discloses real-time interactions with clinical personnel during the treatment in order to: (a) give the model-based prediction of treatment progression and overall treatment outcome; (b) allow clinician to adjust efficacy and safety objectives of the treatment; and (c) stop or otherwise modify the treatment plan.

Another embodiment of the invention discloses automatically manipulation of intensity and location of a focal zone created by an ultrasound phased array in order to deliver a therapeutically-desired thermal dose to the target without violating normal tissue safety.

The present invention has several advantages over the present state of the art in that the present invention may minimize treatment time, has the means for automatic control of safety and efficacy, and offers physician interaction and advisory function.

The present invention also offers several practical and commercial applications over the present state of the art in that it offers invasive and noninvasive thermal therapies, thermally activated targeting of drug delivery, control and modification of blood perfusion, and the controlled breach of brain blood barrier for drug delivery.

Other aspects and features of the invention will become apparent hereinafter.

### BRIEF DESCRIPTION OF DRAWINGS

FIG. 1 is a block diagram representing one embodiment of a feedback control of the thermal dose with explicit normal tissue safety constraints.

FIG. 2(a) depicts a MR-compatible ultrasound positioning system showing the Mylar-covered treatment window, transducer positioning components and a 45° reflecting ultrasound mirror; and FIG. 2(b) depicts a thermal treatment control system during in vivo experiments inside MRI, where the TT controller computes the ultrasound power that leads to the minimum-time treatment without violating normal tissue safety, and sends the result to the function generator and RF amplifier, which provide input to the ultrasound transducer.

FIG. 3(a) depicts the automatically generated ultrasound power applied to a phantom subject; FIG. 3(b) shows an increase in T₉₀, T_{90,ref}, T_{cons} and T_{tum,max} temperatures; and FIG. 3(c) depicts tumor thermal dose evolution during the treatment.

FIG. 4 depicts thermal dose control in in vivo canine, including (a) control input; (b) increase in T₉₀, T_{90,ref}, T_{cons} and T_{tum,max}; and (c) tumor thermal dose.

FIG. 5 depicts spatial distribution of the delivered thermal dose in the treatment domain at various times during canine treatment with the MRI thermometry feedback. The target spans from 9.0 to 10.9 cm. The normal tissue constraint was placed at 8.3 cm.

### BEST MODE(S) FOR CARRYING OUT THE INVENTION

Not meaning to be limited to any particular embodiment, the invention may be illustrated by examples using a magnetic resonance imaging-based (MRI-based) thermal dose controller and an ultrasound transducer as the thermal energy source. However, those of skill in the art would know that the current invention may also include other imaging devices and other thermal energy sources for heating or cooling of the target tissues, and modifications of the described control algorithm that nevertheless preserve the essential features of the invention to automatically control of safety and efficacy of the treatment.

### Thermal Dose Controller

Thermal dose quantifies the relationship between treatment efficacy and target temperature evolution, T(x,t), tε[t₀, t], during therapy, where T(x,t) is the temperature vector taken at position x and time t. A commonly used definition of the thermal dose (D(t)) is the number of cumulative equivalent minutes (CEM) at 43° C.:

$$D ( t ) = CEM at 43 ° C . {T}_{90} = {\int }_{{t}_{0}}^{t} {R}^{[ 43 - {T}_{90} ( τ ) ]} d τ \qquad ( 1 )$$

where T₉₀ is the 10^{th} percentile of the measured temperatures, and R=0 for T₉₀<39° C., R=0.25 for 39<T₉₀<43° C., and R=0.5 for T₉₀≧43° C.

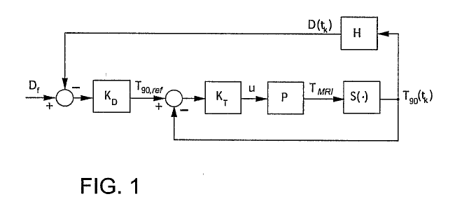

One embodiment of the invention may be an MRI-based thermal dose controller having the cascade structure shown in FIG. 1 and described in Arora et al. 2005b. Briefly, the main nonlinear dose controller K_{D} continuously generates a reference temperature trajectory, T_{90,ref}, for the secondary temperature controller K_{T}. The thermal dose controller is designed to quickly deliver the desired thermal dose without consideration of normal tissue and hardware constraints. It maps the difference between the desired final thermal dose D_{f}, and the already delivered thermal dose D(tₖ), into the reference 10^{th} percentile temperature trajectory, T_{90,ref}:

$$\begin{matrix}{T}_{90 , ref} ( t ) = \frac{1}{ln ( 1 / R )} ln \frac{α ( {t}_{k} )}{{R}^{43}} , & t ∈ [ {t}_{k} , {t}_{k} + Δ t ]\end{matrix} \qquad ( 2 )$$

where α depends on the error between the desired and already delivered thermals dose and the selected final treatment time, t_{f}=tₖ+Δt:

$$α ( {t}_{k} ) = \frac{( {D}_{f} - D ( {t}_{k} ) )}{( {t}_{f} - {t}_{k} )} \qquad ( 3 )$$

The tuning parameter Δt is the moving treatment horizon. In this exemplary embodiment, the vector T(t) (T_{MRI} in FIG. 1) of temperatures in different spatial location inside the subject, P, is measured at the MRI scan rate.

The last measured temperature distribution, T(tₖ), is used to calculate the already delivered thermal dose D(tₖ). The thermal dose error is updated each time a new temperature measurement becomes available, followed by the corresponding update of the reference T_{90,ref} (t). The calculated T_{90,ref}(t) is the reference trajectory for the inner temperature controller. To reduce the treatment time, K_{D} is designed to generate an aggressive reference (by choosing a short treatment horizon Δt) which often cannot be achieved without violating the imposed normal tissue or hardware constraints. The role of K_{T} is to find an ultrasound power, u, such that the difference between T_{90,ref}) and the model-predicted T₉₀(t) is minimized without violating normal tissue and hardware constraints. K_{T} is implemented as a linear, constrained, model predictive controller, which finds m future control moves, u=[u(tₖ, . . . u(tₖ₊ₘ₋₁)], by solving, in real time, the following minimization problem:

$${min}_{u} J ( k ) = {\sum }_{j = 1}^{p} {{w}_{y} ( {t}_{j} ) [ {T}_{90 , ref} ( {t}_{k + j} ) - {T}_{90} ( {t}_{k + j} ) ]}^{2} + {\sum }_{j = 1}^{m} {{w}_{u} ( {t}_{j} ) [ u ( {t}_{k + j - 1} ) ]}^{2} \qquad ( 4 )$$

subject to normal tissue and actuation constraints:

T_{cons}(tₖ₊ⱼ)≦Tₘₐₓjε[1, p] (5)

0≦u(k+j−1)≦uₘₐₓjε[1, m] (6)

where weights w_{y} and wᵤ penalize the error between the desired and the predicted T₉₀ and the control effort, respectively. The normal tissue constraints, Tₘₐₓ, are imposed in terms of the maximum allowable temperature in the selected normal tissue location, T_{cons}. The hardware limitation on the maximum possible transducer power is given by uₘₐₓ. The tuning parameters p and m are the prediction and control horizons. The prediction horizon, p, is chosen long enough to predict the thermal dose accrued during tissue cooling after the power is turned off. The control horizon, m, determines the number of future control moves that are calculated each time a new measurement becomes available. Only the first component, u(k), of the calculated vector u of m sequential power levels is sent to the transducer, and the process is repeated at tₖ₊₁, when the next MRI temperature measurement becomes available. The prediction of T₉₀(t) is calculated as S(T(t)), where the vector of predicted temperature, T(t), must satisfy the thermal response model:

\dot{T}(t)=AT(t)+Bu(t), tε[tₖ₊₁, tₖ₊ₚ] (7)

In one embodiment, the predictive model (7) is obtained by finite difference approximation of a one-dimensional Pennes bioheat transfer equation:

$$pC \frac{\partial T}{\partial t} = k \frac{{\partial }^{2} T}{\partial {x}^{2}} - {W}_{e} {C}_{b} ( T - {T}_{a} ) + Q \qquad ( 8 )$$

where C and C_{b} are the specific heat of tissue and blood [J/(kg° C.)], Wₑ[kg/(m³s)] is the effective blood perfusion parameter, Tₐ is the arterial temperature (in the subject, Tₐ is the temperature before heating is initiated), which was assumed to be constant for the duration of experiments and Q is the power deposition density in W/m³. All results are reported as the deviation of the subject's temperature from the baseline, which was set equal to Tₐ. The values of patient-specific perfusion and power deposition in the Penes model were identified experimentally, following the procedure described elsewhere (Arora et al. 2005c).

The predictive model (7) is used internally by the control system. Matrix A depends on both conduction and perfusion. The term Bᵤ approximates the power deposition term, Q, where u is the applied ultrasound power in Watts. The state T is a vector of deviation temperatures above Tₐ. The position of the ultrasound transducer was fixed and the magnitude of the applied ultrasound power, u, was the only manipulated variable.

The phantom experiments were performed with an 11×11×7 cm agarose phantom. The T2 relaxation time of the phantom was modified by adding one millimole-per-liter of copper sulfate to the recipe of Madsen et al. 1998. After preparation, the tissue-mimicking phantom was allowed to solidify inside of an acrylic box with a Mylar membrane on the bottom surface.

Animal experiments were conducted with a 29 kg male Labrador. The animal was given 75 mg (1.5 ml) of Telazol (Lederle Pharmaceuticals, Carolina, Puerto Rico) by IM injection. When the animal was recumbent, the trachea was intubated. For the duration of the experiment the dog was mechanically ventilated with Isoflurane in oxygen keeping the end tidal CO₂ at approximately 38±2 mm Hg. An intravenous drip was started in the cephalic vein with lactated Ringer's (LR) solution. The dog received ˜15 ml of LR per kg of body weight per hour of anesthesia. The dog was given pancuronium bromide at a rate of 1 mg/hr to inhibit leg motion. Blood pressure was measured with a noninvasive cuff placed on the forelimb. Isoflurane concentration was adjusted to keep the mean blood pressure at about 90±10 mm Hg. The SpO₂ was monitored and maintained at approximately 98% for the duration of anesthesia. Throughout the experiments, the rate of respiration was controlled by a mechanical ventilator. The rate of respiration was set to allow a breathing cycle of six seconds. In order to minimize MRI artifacts due to canine breathing motion and the associated change in susceptibility, the MRI scanner began acquiring an image immediately after each exhalation and completed the scan before the following inhalation. To improve the ultrasound coupling, prior to the experiments the hair on the dog's thigh was removed with clippers and hair removal cream.

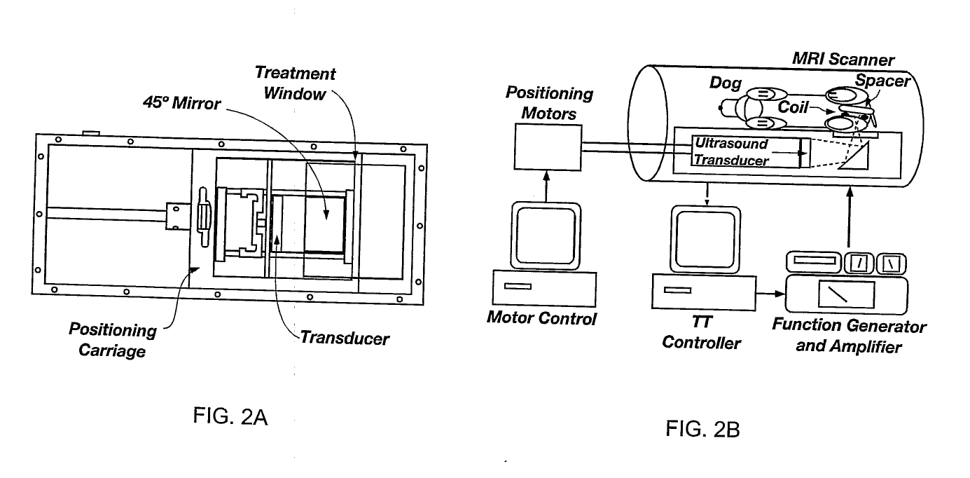

While not meaning to be limited to a single embodiment of the current invention, both phantom and canine experiments were performed using an in-house manufactured Magnetic Resonance Compatible Ultrasound Positioning System (MaRCUPS), depicted in FIG. 2(a). The ultrasound field was generated by a single, stationary, spherically focused, air-backed transducer, resonating at 1.5 MHz, with a diameter and radius of curvature of 10 and 18 cm, respectively. Further details of driving circuitry and MaRCUPS design are given in Arora et al. 2005b.

MR imaging was performed using a Siemens Trio 3T Magnetom scanner. To improve the signal-to-noise ratio (SNR) and thus allow for a faster scan rate, prior to the canine and phantom experiments a custom built receive-only surface coil was tuned and matched to the desired imaging location. The temperature change in the dog's thigh and the phantom were measured using the proton resonance frequency (PRF) shift method with a temperature coefficient of 0.01 ppm/° C. Image data were gathered using a gradient echo (GRE) pulse sequence with a spoiled gradient. The following parameters were used for temperature measurements in the phantom during control experiments: TR=14 ms, TE=10, voxel size=2.0×4.0 x 3.0 mm, FOV=256 mm, matrix size=128×64, flip angle=25°, and scan time of 1.15 seconds with a phase resolution of fifty percent to reduce acquisition time. During model identification step tests, the parameters were kept the same except that the scan was taken with the repetition time of TR=30 ms and the corresponding overall scan rate of 2.45 seconds. The data were zero-filled in the phase encoding direction to a matrix of 128×128.

In the canine thigh, the temperature during model identification step tests and closed-loop controller runs was imaged using TR=40 ms, TE=10 ms, 1.6×3.2 x 3.0 mm voxel size, 200 mm FOV, 128×64 matrix, 25° flip angle, and 2.56 seconds scan time with a phase resolution of fifty percent. The data were zero-filled in the phase encoding direction to a matrix of 128×128. Fat saturation was applied to the GRE sequence to suppress the fat signal and improve the SNR A delay of 3.4 seconds was added to the data acquisition sequence, which made the overall rate at which data were acquired equal to 5.96 seconds. This synchronized temperature imaging with the breathing cycle and thus reduced motion artifacts.

In each case, the subject (phantom or a dog) was positioned on MaRCUPS in the center of the Mylar treatment window (FIG. 2(b)) with the receive coil in the sagittal plane. Ultrasound gel was used to couple the subject to the Mylar window. The focal zone of the transducer was located by applying a step input of ultrasound power while phase images were acquired in the coronal plane, approximately halfway through the subject. A sagittal MR thermal image corresponding to maximum temperature location was chosen through the center of the heated region. To ensure that the center of the ultrasound beam was located in the chosen since, the step input of power and phase image subtraction were repeated while slightly adjusting the position of the sagittal slice.

### EXAMPLES

Not meaning to be limited by a single embodiment of the invention, the following examples disclose a MRI-based thermal controller using an ultrasound transducer as the thermal energy source. However, those of skill in the art would know that the current invention may also include other imaging devices and other thermal energy sources.

The following examples were carried out with the efficacy objective of delivering the specified CEM43° T₉₀ to the designated tumor region while maintaining normal tissue temperature in the selected location below the specified maximum allowable value (safety objective). A number of phantom and canine example runs were performed to analyze the effect of the tuning parameters on controller performance. While in vitro and in vivo results are disclosed herein, those of skill in the art will know the invention may be used for other thermal therapies. It was found that the effect of controller tuning qualitatively agrees with the previous conclusions, obtained using computer simulations (Arora et al. 2005a) and experiments with thermocouple-based feedback (Arora et al. 2005b).

### Example 1

### Phantom Results

The objective was to deliver D_{f}=10 CEM43° T₄₀ to the selected target region while limiting the temperature at the constraint location to below 6.5° C. A low thermal dose was selected to reduce time and the associated cost required to complete multiple experimental runs. The ultrasound power was constrained (uₘₐₓ=11 W) to reflect hardware limitations and to avoid cavitation.

The controller tuning parameters were set as follows: prediction horizon, p=4.6;

control horizon, m=2.3 seconds; and the moving treatment horizon, t_{TH}=4.6 seconds. The treatment horizon was selected to: (a) force the activation of the transducer power constraint at the beginning of the treatment when the normal tissue temperature constraint was not active, and (b) towards the end of the treatment, to ensure that the thermal dose controller, K_{D}, generates an almost attainable reference temperature trajectory to minimize overdosing of the target.

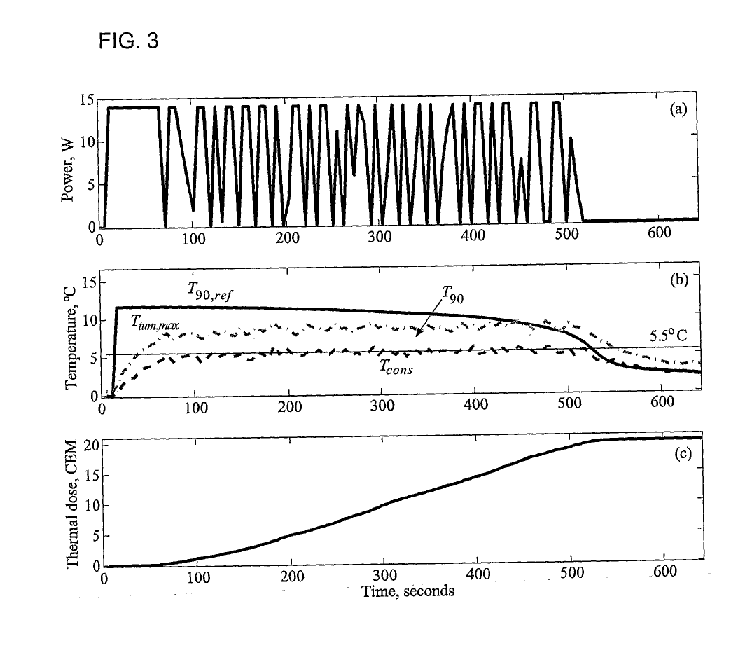

FIG. 3 depicts the evolution of the controlled power output, u; maximum temperature increase inside the target, T_{tum, max}; T₉₀ of the target; the temperature at the constraint location, T_{cons}; the reference temperature generated by the outer controller, T_{90,ref}, and the thermal dose in the target. All temperatures are plotted as deviations above the baseline value.

FIG. 3(b) indicates that at t=103 seconds, the temperature constraint became active. The constrained model predictive controller, K_{T}, automatically reduced the ultrasound power (FIG. 3a) in such a way that temperature in the constraint location is maintained near the maximum permitted value of 6.5° C.

The selected small value of the moving treatment horizon (t_{TH}=4.6 seconds) resulted an aggressive thermal dose controller, K_{D}. The K_{D}-generated reference trajectory (T_{90,ref} in FIG. 3b) is aggressive. In an attempt to follow T_{90,ref} as close as possible, the constrained temperature controller, K_{T}, causes the saturation of ultrasound power at the beginning of the treatment. When the temperature in the constrained location reaches the maximum allowable level, the temperature controller begins to automatically modulate the ultrasound power so that the treatment progresses very close to the normal tissue constant. The progression of the treatment close to a constraint is necessary to achieve the efficacy objective in minimum time. Towards the end of the treatment, as the delivered thermal dose approaches the desired value, D_{f}, the reference T_{90, ref}(t) drops to zero as the error, Df−D(tₖ)→0. After the power is switched off, the residual thermal dose is delivered during tissue cooling. The desired thermal dose of 10CEM43° T₉₀ was achieved in the target at approximately t=330 seconds. Either the temperature or the power constraint is active throughout the treatment, which for the case of a single stationary transducer, provides conditions for minimum-time treatment.

### Example 2

### In Vivo Canine Results

The in vivo results were obtained with the ultrasound power constrained to uₘₐₓ=14 W. The desired final thermal dose was set to 20 CEM. Compared to the Example 1 phantom case, a tighter and clinically more realistic normal tissue constraint of 5.5° C. was imposed in the close proximity of the target. By minimizing tissue damage, it was possible to perform multiple tests with the same subject and evaluate the effect of various factors on the performance of the automatic treatment control system.

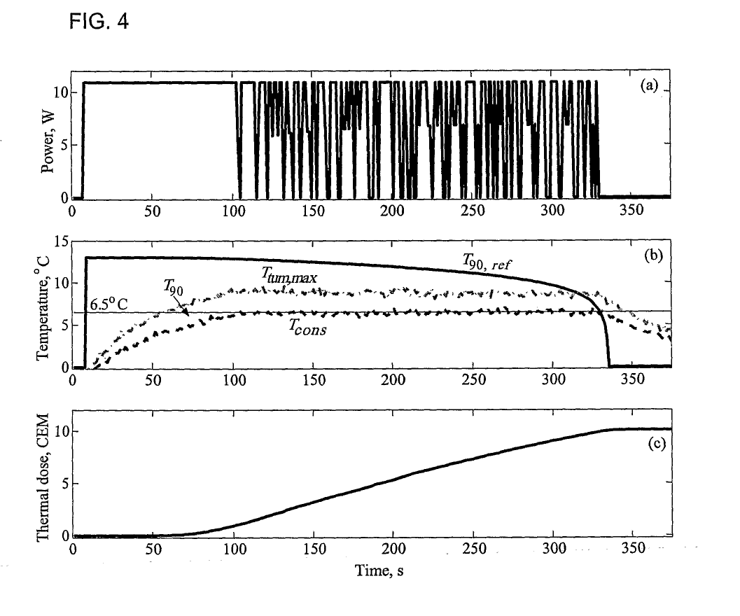

FIG. 4 depicts the controller-generated power, MR temperature measurements, and the resulting thermal dose for one of the test runs. The controller tuning parameters p and in were set to 24 and 12 seconds, respectively. The value of the moving treatment horizon, t_{TH}, was set to 24 seconds, which forced the activation of the transducer constraint at the beginning of the treatment. Because of a slower sampling of MR-thermometry measurements during in vivo experiments (5.96 seconds vs. 1.15 seconds in phantom case), the treatment controller was tuned less aggressively compared to the phantom case by selecting larger values of the treatment, control and prediction horizons. FIG. 4(b) shows that at t=70 seconds the temperature constraint became active. To avoid constraint violation, the temperature controller, K_{T}, changed the ultrasound power in such a way that temperature in the constraint location (FIG. 4b) was maintained close to the value of 5.5° C. to minimize the treatment time. The highest safe temperature in the selected normal tissue location was maintained with active modulation of the applied power (FIG. 4a), which is an expected behavior for an aggressively tuned controller. T_{cons}(t) is shown in FIG. 4(b), where the reference, T_{90,ref}, and the measured T₉₀ are also shown. FIG. 4(c) indicates that the desired thermal dose of 20 CEM was achieved in the target in approximately 515 seconds.

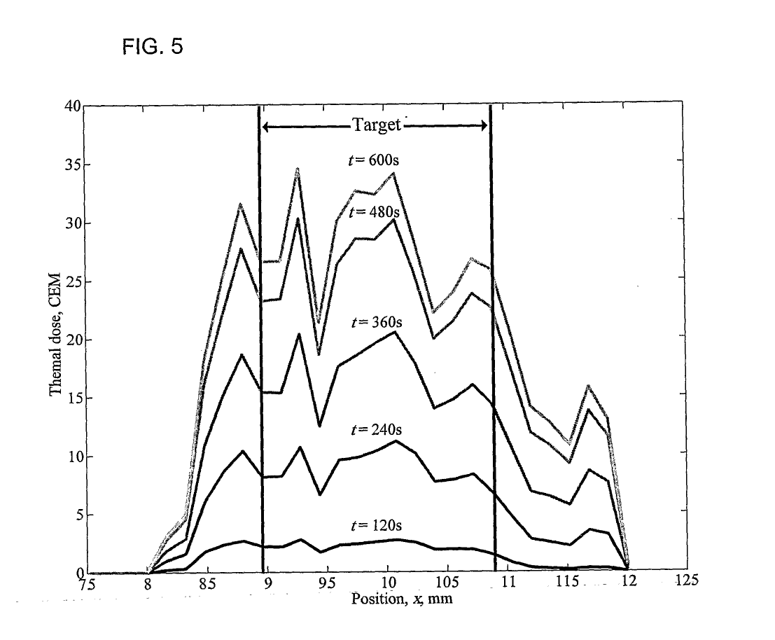

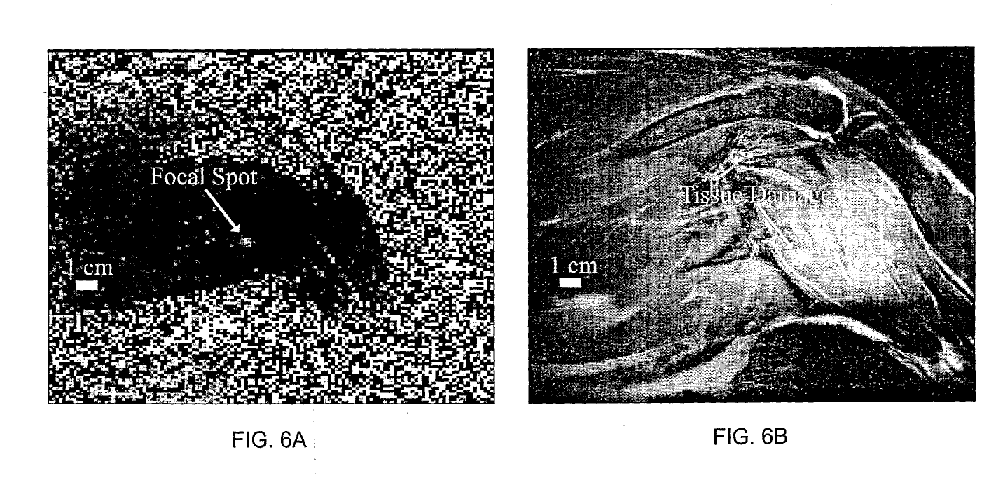

FIG. 5 depicts the spatial distribution of thermal dose, D(x), on the line of beam symmetry of focused ultrasound transducer at various times during the treatment. A sharp thermal dose delineation in the target and normal tissue at the constraint location, x=8.3 cm, is evident.

During additional experiment runs (results not shown), an even lower value of normal tissue constraint (4° C.) was used. This further reduced the thermal dose delivered at the constraint location, but at the expense of considerable lengthening of the treatment time. Such correlation between the treatment duration and the imposed normal tissue constraints is an expression of the tradeoff between efficacy and safety objectives: when safety requirements are relaxed, the efficacy can be achieved with a more aggressive and shorter treatment. The treatment time was also longer when the controller was de-tuned (i.e., made less aggressive) by using larger values of the treatment horizon, t_{TH}, which is the most important tuning parameter of the implemented control system.

Example 1 and Example 2 demonstrate the feasibility of automatic, MRI-based control of minimum-time, safe and efficient thermal therapies. The disclosed control system simultaneously achieves the specified efficacy and safety objectives, expressed in terms of the desired thermal dose in the target and the maximum allowed normal tissue temperatures in the clinician-selected locations. The current invention, validated using stationary ultrasound actuation, can be used without modifications with different stationary actuation modalities, including radio frequency, microwave and laser treatments, performed with noninvasive, interstitial or intracavitary applicators.

The disclosed control system automatically delivers the desired target thermal dose in the presence of temporally and spatially varying temperatures. Since the control problem is formulated in terms of thermal dose, the controller does not try to create a uniform temperature distribution in the target, as is often attempted in standard hyperthermia by utilizing highly specialized, site- and patient-specific applicators. Instead, the treatment is controlled by directly and automatically controlling the thermal dose delivered to the target, subject to normal tissue constraints. This approach is equally applicable to moderate-temperature hyperthermia and high temperature thermal ablation, as long as the appropriate thermal dose is specified by the user. In thermal ablations, the automatic handling of normal tissue constraints allows us to minimize the treatment time by implementing the most aggressive treatment that does not violate the safety objectives.

In one embodiment, the capability of the current invention to safely deliver the specified thermal dose may be enhanced by the use of MR thermometry which provides spatially distributed measurements of temperatures and reduces treatment uncertainty compared to the case when limited pointwise temperature measurements are used. Another benefit of MR-thermometry may be the improved accuracy of the identified model used internally by the treatment control. Comprehensive MR measurements of temperature distribution may eliminate the need for temperature estimation (generated, for example, by the Kalman filter, as in Arora et al. 2005a), thus reducing the uncertainty in real-time assessment of treatment progression.

The model predictive capability of the current invention may allow it to assess the effect and interaction of m control actions over p steps into the future (p≧m). The thermal dose delivered during tissue cooling is also taken into account by the disclosed model-based controller, which minimizes target overdosing and the active heating time. The disclosed predictive thermal model, internally used by the control system, may be updated each time a new MR temperature measurement becomes available. The continuous model adaptation my decrease the sensitivity of the control system to modeling errors and changing target properties, including blood perfusion and ultrasound absorption.

Furthermore, FIGS. 3(a) and 4(a) show that the treatment evolves with either normal tissue or power constraints active at all times and may allow for a minimum time treatment. The normal tissue constraint may be kept close to the maximum allowed value with active power modulation. The observed rapid change in the manipulated variable is typical of aggressive, minimum-time, controllers when time-varying disturbances affect the treatment. Earlier simulations (Arora et al. 2005a) showed that in an ideal case of no plant-model mismatch, and with time-invariant disturbances, the controller was able to arrive at the exact power level that maintains the normal tissue at the constrained value, completely eliminating the rapid change of ultrasound power. Note, however, that rapidly changing power causes relatively small temperature variations. If desired, the rapid power change may be impeded by using a larger value of the tuning parameter wᵤ in objective function J, equation (4). However, a higher control penalty will generally lead to a longer than time-optimal treatment. In order to obtain near minimum-time results, wᵤ may be set to zero.

The spatial distribution of the thermal dose in the treatment region, FIG. 5, shows a sharp delineation between the thermal dose in the target and the constraint location in normal tissue. This effectively demonstrates that by imposing temperature constraints, the dose delivered to the surrounding normal tissue may be limited. During all experimental runs, including the case shown in FIG. 4, the CEM43° T₉₀ thermal dose delivered to the target was almost exactly equal to the specified reference value, D_{f}. The corresponding pointwise thermal dose exceeds D_{f} in most spatial locations (as can be seen in FIG. 5). A pointwise overdosing inside the target is usually acceptable from the clinical perspective, and may be expected when the treatment objective is formulated in terms of T₉₀ temperature. A more uniform spatial dose profile and further reduction in treatment times may be possible when additional degrees of freedom are available for automatic control, as in the case when both the ultrasound intensity and the position of the focal zone are controlled in real time.

With little modification, the present invention may be extended to the treatment of large tumors with scanning or phased power fields by subdividing the tumor into subregions, determined by the size and shape of the heating pattern, and their sequential treatment to the desired thermal dose under the control of the described system. The sequence of subregions may be obtained as a result of pre-treatment optimization, or by following a preselected focal zone trajectory (e.g., rastering, as in Hynynen et al. 2001). The heating interaction in different subregions may be accounted for by adapting the thermal dose set point to reflect the already delivered dose due to SAR (specific absorption rate) overlap and heat transfer between subregions. During the entire thermal dose therapy the controller may automatically adjust the power, such that normal tissue constraints are not violated, no matter the location of the focal zone. The current invention may also include the development of a control system which automatically manipulates the focal zone location, rather than relying on pre-specified sequence of positions or trajectories, selected prior to the treatment.

The treatments in Examples 1 and 2 were no longer than 10 minutes. During longer treatments, characteristic of the traditional hyperthermia, the PRF-based MR-thermometry may be susceptible to temporal and spatial variations due to drift of the Bₒ field. Uncertainties in the MR temperature measurements, including those caused by inhomogeneity of susceptibility, may have a negative effect on the ability to achieve safety and efficacy objectives of the treatment. In such cases, the signal correction techniques, utilizing direct measurements at regions with defined phase under ideal conditions (e.g., in a water bolus), as illustrated in Gellermann et al. 2004, may be used to improve the accuracy of MR thermometry.

### Example 3

### Automatic Control of Focal Trajectory and Intensity of Ultrasound Phased Arrays

A prototype treatment control system that automatically selects location and intensity of the ultrasound focal zone to deliver the prescribed thermal dose to the target in minimum time without violating explicitly imposed normal tissue safety constraints is developed. The results of its initial evaluation in a computer-simulated treatment of a realistic three-dimensional breast cancer patient are reported in Niu et al. 2006. These results illustrate salient features of the developed prototype, which are necessary to minimize the treatment duration while simultaneously satisfying the normal tissue safety constraints.

### REFERENCES

The following references are specifically incorporated in their entirety herein by reference:
Arora D., D. Cooley, T. Perry, M. Skliar and R. B. Roemer. Direct thermal dose control of constrained focused ultrasound treatments: phantom and in vivo evaluation. Phys. Med. Biol. 2005a; 50:1919-35.
Arora D., M. Skliar and R. B. Roemer. Minimum-time thermal dose control of thermal therapies. IEEE Trans. Biomed. Eng. 2005b; 52:191-200.
Arora D., D. Cooley, T. Perry, J. Guo, D. Parker, M. Skliar and R. B. Roemer. Thermal dose control of ultrasound therapies using MR thermometry images: and in vitro phantom study. Proc. 2005 American Control Conf. 2005c; 405-10.
Arora D., M. Skliar, D. Cooley, A. Blankespoor, J. Moellmer and R. B. Roemer. Nonlinear model predictive thermal dose control of thermal therapies: experimental validation with phantoms. Proc. 2004 American Control Conf. 2004; 1627-32.
Arora D., M. Skliar and R. B. Roemer. Nonlinear and model predictive control of thermal dose in high temperature therapies. Proc. 2003 American Control Conf. 2003; 1200-05.
Arora D., M. Skliar and R. B. Roemer. Model predictive control of ultrasound hyperthermia treatments of cancer. Proc. 2002 American Control Conf. 2002; 2897-2902.
Arora D., D. Cooley, T. Perry, J. Guo, A. Richardson, J. Moellmer, R. Hadley, D. Parker, M. Skliar and R. B. Roemer. MR Thermometry-based feedback control of efficacy and safety of minimum time thermal therapies: phantom and in vivo evaluations. Int. J. Hyperthermia, 22:29-42, 2006.
Bornsetin B. A., P. S. Souranjian, J. L. Hansen, S. M. Fraser, L. A. Gelwan, G. A. Teicher and G. K. Svensson. Local hyperthermia, radiation therapy and chemotherapy in patients with local regional recurrence of breast carcinoma. Int. J. Rad. Oncol. Biol Phys. 1993; 25:79-85.
Gellermann J., W. Wiodarctzyk, H. Ganter, J. Nadobny, H. Fahling, M. Seebass, R. Felix, and P. A. Wust. Practical approach to thermography in a hyperthermia/magnetic resonance hybrid system: Validation in a heterogeneous phantom. Int. J. Oncology Biol. Phys. 2004; 61:267-277.
Guthkelch A. N., L. P. Carter, J. R. Cassady, K. H. Hynynen, R. P. Iacono, P. C. Johnson, E. A. Obbens, R. B. Roemer, J. F. Seeger and D. S. Shimm. Treatment of malignant brain tumors with focused ultrasound hyperthermia and radiation: results of phase I trial. J. Neuro. Oncol. 1991; 10:271-84.
Hynynen K. The threshold for thermally significant cavitation in dog's thigh muscle in vivo. Ultrasound Med. 1991; 17:157-69.
Hynynen K., O. Pomeroy, D. N. Smith, P. E. Huber, N. J. McDannold, J. K. Kettenbach, J. Baum, S. Singer, and F. A. Jolesz. MR imaging-guided focused ultrasound surgery of fibroadenomas in the breast: A feasibility study. Radiology. 2001; 219:176-185.
Madsen E. L., G. R. Frank and F. Dong. Liquid or solid ultrasonically tissue-mimicking materials with very low scatter. Ultrasound in Med. & Biol. 1998; 24:535-542.
Niu R., A. Blankespoor, J. Moellmer, R. Roemer and M. Skliar. Automatic control of focal trajectory and intensity of ultrasound phased arrays in minimum-time delivery of thermal dose with imposed normal tissue constraints. Proceedings of the 6th International Symposium on Therapeutic Ultrasound, Oxford, UK.
Palussiere J., R. Salomir, B. Le Bail, R. Fawaz, B. Quesson, N. Grenier and T. W. Moomem. Feasibility of MR-guided focused ultrasound with real-time temperature mapping and continuous sonication for ablation of VX2 carcinoma in rabbit thigh. Magn. Reson. Med. 2003; 49:89-98.
Perry T. Use of a magnetic resonance compatible ultrasound positioning system for SAR field estimation. Masters Thesis 2005 University of Utah.
Poissonnier L., A. Gelet, J. Y. Capelon, R. Bouvier, O. Rouviere, C. Panguad, D. Lyonnet and J. M. Dubernard. Results of transrectal focused ultrasound for the treatment of localized prostate cancer. Prog. Urol. 2003; 13:60-72.
Uchida T., N. T. Sanghvi, T. A. Gardner, M. O. Koch, D. Ishii, S. Minei, T. Satoh, T. Hyodo, A. Irie and S. Baba. Transrectal high-intensity focused ultrasound for treatment of patients with stage T1b-2n0m0 localized prostate cancer: a preliminary report. Urology 2002; 59:394-8.
Wu F., W. Z. Chen, J. Bai, Z. L. Wang, H. Zhu and Z. B. Wang. Pathological changes in human malignant carcinoma treated with high-intensity focused ultrasound. 2001 Ultrasound Med. Biol. 2001; 27:1099-106.

What is claimed is:

1. A thermal treatment control system, comprising:
an imaging device for specifying a treatment target's geometry and/or location;
a thermal energy element for applying a thermal treatment wherein the thermal treatment comprises heating and/or cooling of a target for therapeutic purposes;
a thermal energy element for applying cooling and/or heating of normal tissues to prevent or minimize normal tissue damage outside the treatment target;
a thermal energy detecting element for detecting a measured tissue response to the thermal treatment; and
a feedback controller for a real-time modification of the intensity and spatial distribution of a thermal energy created by the thermal energy element in order to achieve the efficacy objectives of the therapy in a minimum or reduced time while simultaneously satisfying normal tissue safety constraints;
wherein the measured tissue response to the thermal treatment is used as feedback by the feedback controller and the real-time modification is made to the operation of the thermal energy element in reaction to the measured tissue response.

2. The thermal treatment control system of claim 1, wherein a prescribed target treatment efficacy in terms of a thermal dose, a temperature distribution, a temperature change, or other treatment parameters is specified for achieving a desired treatment outcome.

3. The thermal treatment control system of claim 1, wherein treatment conditions are specified for ensuring the safety of the tissues outside a treatment target and wherein the treatment parameter is in the form of temperature, temperature change, thermal dose, or limits on thermal response of the normal tissues outside the treatment target.

4. The thermal treatment control system of claim 1, wherein a hardware constraint of the thermal treatment system or thermal element is specified.

5. The thermal treatment control system of claim 1, wherein the thermal energy element for applying a thermal treatment comprises a noninvasive power delivery element.

6. The thermal energy element of claim 5, further comprising single or multiple transducers or transducer arrays for ultrasound heating, radio frequency heating, and/or microwave heating.

7. The thermal treatment control system of claim 1, wherein the thermal energy element for applying a thermal treatment comprises an invasive power delivery element.

8. The thermal energy element of claim 7, further comprising an interstitial microwave, radio frequency and/or optical needles and applicators.

9. The thermal energy element of claim 7, further comprising an interstitial ultrasound element.

10. The thermal treatment control system of claim 1, wherein the thermal energy detecting element for detecting a measured tissue response to the thermal treatment comprises a noninvasive thermal energy detecting element.

11. The thermal treatment control system of claim 10, further comprising means for taking a magnetic resonance temperature measurement.

12. The thermal treatment control system of claim 1, wherein the thermal energy detecting element for detecting a measured tissue response to the thermal treatment comprises an invasive thermal energy detecting element.

13. The invasive thermal energy detecting element of claim 12, further comprising an invasive thermal energy detecting probe.

14. The thermal treatment control system of claim 1, wherein the thermal energy element for applying a thermal treatment is selected from the group consisting of a single stationary ultrasound transducer, a single stationary interstitial microwave, radio frequency and/or optical needle or applicator, a single transducer which may be repositioned by mechanical or other means, multiple stationary ultrasound transducers comprising a stationary phased array of individually controlled ultrasound transducers, multiple stationary microwave, radio frequency and/or optical needles and applicators, multiple ultrasound transducers which may be repositioned by mechanical or other means, stationary and repositionable ultrasound transducers, stationary and repositionable microwave, radio frequency and/or optical needles and applicators, and any combination thereof.

15. The thermal treatment control system of claim 1, wherein the feedback controller includes a predictive thermal model;

16. The thermal treatment control system of claim 15, wherein the predictive model includes a transducer model.

17. The thermal treatment control system of claim 15, wherein the feedback controller is an adaptive treatment controller which re-identifies the predictive thermal model and transducer models, and utilizes the re-identified models in automatic control of the thermal therapy.

18. The thermal treatment control system of claim 1, further comprising: means for providing a real-time interaction between the thermal treatment control system and at least one clinical personnel during the thermal treatment.

19. The thermal treatment control system of claim 18, wherein the means for providing the real-time interaction uses model-based prediction of treatment progression and/or treatment outcome to change the thermal treatment.

20. The thermal treatment control system of claim 18, wherein means for providing the real-time interaction adjusts the efficacy and safety objectives of the thermal treatment based on the treatment monitoring and the model-based prediction of treatment progression and/or treatment outcome.

21. The thermal treatment control system of claim 3, wherein a positional constraint for normal tissues outside the treatment target is specified.

22. The thermal treatment control system of claim 1, further comprising a data processing means that coordinates the actions of the imaging device, the thermal energy element, the thermal energy detecting element, and the feedback controller.

23. The thermal treatment control system of claim 1, wherein the measured tissue response to the thermal treatment is in the form of a temperature change and the temperature change is measured by using a proton resonance frequency shift method.

24. A method for thermal treatment of a subject, the method comprising:
specifying a treatment target's geometry and/or location using an imaging device;
applying a thermal treatment wherein the thermal treatment comprises heating and/or cooling of a target tissue for therapeutic purposes;
applying a thermal treatment wherein a thermal energy element applies cooling and/or heating of normal tissues to prevent or minimize normal tissue damage outside the treatment target;
detecting a measured tissue response to the thermal treatment; and
modify the intensity and spatial distribution of a thermal dose using a real-time feedback controller element in order to achieve the efficacy objectives of the therapy in a minimum or reduced time while simultaneously satisfying normal tissue safety constraints;
wherein the measured tissue response to the thermal treatment is used as feedback by the feedback controller and the real-time modification is made to the operation of the thermal energy element in reaction to the measured tissue response.
